(19) United States
(12) Patent Application Publication
(10) Pub. No.: US 2025/0381365 A1
(43) Pub. Date: Dec. 18, 2025

(54) DIGITAL SOUNDSCAPES FOR AUGMENTING PSYCHOTROPIC THERAPIES

(71) Applicant: PsySonics P.B.C., Asheville, NC (US)

(72) Inventor: Frank Rogers Masson, Asheville, NC (US)

(21) Appl. No.: 19/235,596

(22) Filed: Jun. 12, 2025

Related U.S. Application Data
(60) Provisional application No. 63/660,421, filed on Jun. 14, 2024.

(51) Int. Cl. A61M 21/02; A61K 31/135; A61K 31/36; A61K 31/675; A61M 21/00; G10L 21/043; H04S 7/00

(52) U.S. Cl. CPC A61M 21/02; A61K 31/135; A61K 31/36; A61K 31/675; G10L 21/043; H04S 7/307; A61M 2021/0033; A61M 2021/0038; A61M 2021/0077

(57) ABSTRACT

Disclosed herein are a digitally produced soundscape for augmenting therapeutic neurological effects of a psychotropic substance, as well as a method of distributing the substance and a psychotropic therapy method including providing the soundscape. The soundscape lacks traditional musical elements such as identifiable musical instrumentation, identifiable vocalizations, and identifiable harmonic structure. The soundscape includes a fundamental frequency associated with at least one of blood pressure reduction, heart rate reduction, or a sensation of safety.

### CROSS-REFERENCE TO RELATED APPLICATION(S)

This application claims priority to and the benefits of U.S. Provisional Application No. 63/660,421, titled “SYSTEM AND METHOD FOR CREATING PSYCHOACOUSTIC SOUND FOR PSYCHEDELIC-ASSISTED THERAPY” filed on Jun. 14, 2024. The content of the aforementioned application is herein incorporated by reference in its entirety.

### TECHNICAL FIELD

This disclosure relates to the field of psychoacoustics and therapeutic sound engineering and design, specifically tailored for use within psychotropic therapy.

### BACKGROUND

Psychotropic therapy is the use of psychotropic substances, or substances that affect the mind, emotions, and behavior, to treat mental health conditions such as depression, anxiety, bipolar disorder, and schizophrenia. Psychotropic therapy has gained recognition as a powerful therapeutic tool for various mental health conditions and has become more widely adopted in recent years.

Digital audio production refers to the process of creating, recording, editing, mixing, and mastering sound using digital technology. Digital audio production is often performed using a digital audio workstation (DAW), which is a specialized software application or integrated hardware system used for digital audio production. DAWs include various pre-installed tools for manipulating sounds and are typically compatible with various plugins or additional tools that can be added to the DAW to perform sound manipulation functions.

### BRIEF DESCRIPTION OF THE DRAWINGS

FIG. 1 is an example soundscape distribution environment for providing a soundscape to a patient undergoing psychotropic therapy, in accordance with some embodiments of the present technology.

FIG. 2 an example binaural audio plugin that may be used to render the soundscape as a binaural audio file.

FIG. 3 is an example equalizer (EQ) setting that may be applied to the soundscape, in accordance with some embodiments of the present technology.

FIG. 4 is another example EQ setting that may be applied to the soundscape, in accordance with some embodiments of the present technology.

FIG. 5 is an example compressor setting that may be applied to the soundscape, in accordance with some embodiments of the present technology.

FIG. 6 is an example saturation setting that may be applied to the soundscape, in accordance with some embodiments of the present technology.

FIG. 7 is an example peak limiter setting that may be applied to the soundscape to limit the peak volume of sounds in the soundscape, in accordance with some embodiments of the present technology.

FIG. 8 is an example reverb setting that may be applied to the soundscape, in accordance with some embodiments of the present technology.

FIG. 9 is another example reverb setting that may be applied to the soundscape, in accordance with some embodiments of the present technology.

FIG. 10 is an example delay setting that may be applied to the soundscape, in accordance with some embodiments of the present technology.

FIG. 11 is a flowchart depicting an example method of providing a soundscape to a patient, in accordance with some embodiments of the present technology.

FIG. 12 is a flowchart depicting an example psychotropic therapy method, in accordance with some embodiments of the present technology.

### DETAILED DESCRIPTION

Despite psychotropic therapy gaining recognition as a powerful therapeutic tool for various mental health conditions, the ability of sound to enhance existing psychotropic therapy techniques is often overlooked or insufficiently addressed. Some music or soundscapes designed to complement existing psychotropic therapies do exist, but typically do not effectively enhance or increase the effectiveness of the therapeutic process or facilitate the desired mental healing, as these solutions do not include or sufficiently emphasize frequencies that are soothing to the human ear, evoke feelings of safety, and/or contribute to reducing physiological stress responses, such as heightened blood pressure or heart rate.

Additionally, existing solutions often include traditional musical elements such as harmonic structures, identifiable rhythms, identifiable tempos, identifiable instrumentation/vocalizations, or genre characteristics. While valuable in other audio listening contexts, these traditional musical elements may distract a patient undergoing psychotropic therapy, diverting attention away from the overall therapeutic experience and focusing it on the features of the music or soundscape itself. For example, because humans are physiologically predisposed to process sharp, bright, and/or quick transient vibrations with immediacy, sounds like percussion, plucked or strummed guitar, sitar, or piano may be disruptive during a psychotropic therapy session. This distraction/disruption, along with the lack of effectively emphasized frequencies in existing solutions, ultimately limits their effectiveness for enhancing the treatment of mental health conditions during a psychotropic therapy session. Therefore, there is a need for a novel approach to creating soundscapes better tailored to psychotropic therapy sessions.

The present disclosure addresses these limitations of existing music and soundscapes for augmenting existing psychotropic therapies by providing a soundscape designed by combining principles of audio engineering, psychoacoustics, neuroscience, and therapeutic intent to augment various psychotropic therapies. The soundscape includes fundamental frequencies found in nature, along with specifically designed, selected, and engineered fundamental frequencies to create a sonic environment configured to help induce and support therapeutic states of consciousness that occur when the neurological effects of a psychotropic substance are experienced by a patient. These frequencies may be selected based on their known therapeutic effects, including relaxation, introspection, and altered states of consciousness. In some embodiments, the soundscape includes a fundamental frequency associated with at least one of blood pressure reduction, heart rate reduction, or a sensation of safety. Including such a fundamental frequency allows the soundscape to directly enhance the positive neurological experiences and reduce the stress responses induced by psychotropic substances more effectively than existing solutions, ultimately resulting in a more productive therapeutic experience.

Additionally, the soundscape is intentionally designed to lack traditional musical elements that may distract or otherwise disrupt a patient during psychotropic therapy. For example, the soundscape may lack identifiable musical instrumentation, identifiable vocalizations, identifiable harmonic structure, identifiable rhythm, and/or identifiable tempo. Excluding these features from the soundscape enables a patient experiencing neurological effects of a psychotropic substance to listen to the soundscape and experience the therapeutic enhancements of its carefully chosen frequencies without being distracted from the therapeutic experience. Furthermore, excluding traditional musical elements makes the soundscape more likely to be effective for a variety of patients who may have different relationships to different types of music and/or may be triggered by certain sounds.

Instead of traditional music elements, the soundscape includes crafted sounds, which may be digital manipulations of natural sounds or instruments, that are chosen to complement and harmonize with the fundamental frequency or frequencies of the soundscape. These sounds are composed and layered to create a cohesive sonic journey without the use of traditional musical elements, supporting the patient throughout different stages of a psychotropic therapy session without disruption or distraction. Binaural rendering techniques and/or other psychoacoustic techniques may be used to further enhance the potential of the soundscape to promote relaxation, focus, and emotional release in the patient. The resulting soundscape may be encoded in high-quality immersive audio formats suitable for playback in therapeutic settings, and may be customized to complement the neurological effects of particular psychotropic substances, such as 3,4-Methylenedioxymethamphetamine (MDMA), ketamine, and psilocybin.

One example use of the technique disclosed here is within a controlled clinical setting where clinicians are conducting psychotropic therapy sessions for individuals with treatment-resistant depression. Prior to the session, the clinician selects a predesigned soundscape tailored to the psychotropic substance the patient will be taking. The soundscape incorporates frequencies associated with emotional healing and self-reflection. During the session, the patient listens to the soundscape through a binaural experience with headphones or within an immersive surround sound environment (e.g., Dolby® ATMOS®), allowing the therapeutic soundscape to accompany and support the inner exploration and healing process of the patient. As the session progresses, the soundscape maintains a consistent presence and supports the patient's evolving experience, providing an immersive auditory environment conducive to positive therapeutic outcomes.

### Example Soundscape Distribution Environment

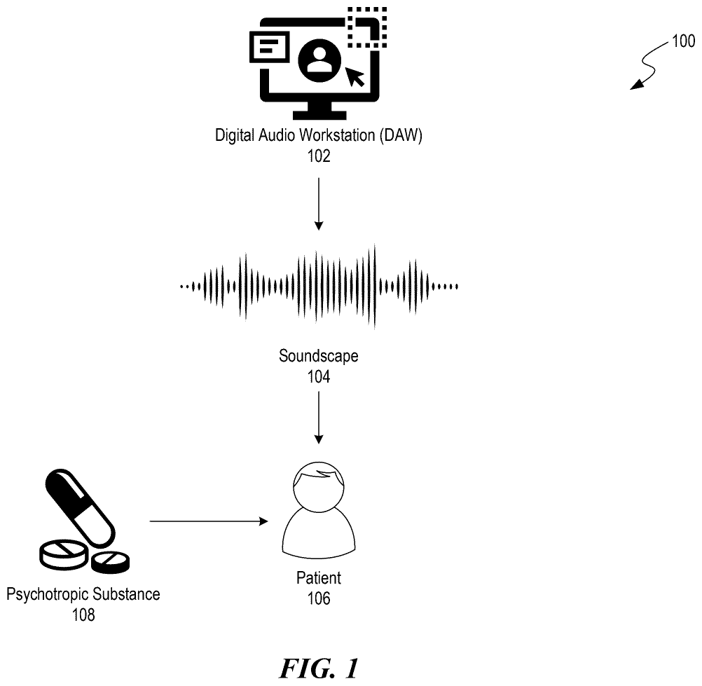

FIG. 1 is an example soundscape distribution environment 100 for providing a soundscape 104 to a patient 106 undergoing psychotropic therapy, in accordance with some embodiments of the present technology. The soundscape distribution environment 100 includes a digital audio workstation (DAW) 102, the soundscape 104, the patient 106, and a psychotropic substance 108. It will be appreciated, however, that some implementations of the soundscape distribution environment 100 may include different and/or additional components or may include components connected in different ways.

The DAW 102 is a software application and/or an integrated hardware system used for digital audio production in which the soundscape 104 is digitally produced. For example, the DAW 102 may be Avid® Pro Tools®, Apple® Logic Pro®, or another commercially available DAW. The soundscape 104 is an audio file lacking one or more traditional musical elements, such as identifiable musical instrumentation (e.g., sounds that are identifiable as being produced by a particular instrument), identifiable vocalizations (e.g., sounds that are identifiable as a human voice), identifiable harmonic structure (e.g., identifiable musical expression created by multiple different sounds), identifiable rhythm, and/or identifiable tempo. “Identifiable” in this context refers to being recognizable by a listener of average hearing and familiarity with music. Excluding one or more of these traditional musical elements from the soundscape 104 enables the patient 106 to listen to the soundscape 104 during a psychotropic therapy session and experience one or more desired therapeutic effects (described below) from the listening without being distracted from the therapeutic experience. Furthermore, excluding these traditional musical elements makes the therapeutic effect of the soundscape 104 more likely to apply to a variety of patients who may have different relationships with and/or reactions to identifiable musical features. In some embodiments, the soundscape 104 is a stereo WAV file with a 24-bit depth and a 96k sample rate or another high-resolution audio file.

Using the DAW 102, a fundamental frequency associated with a desired therapeutic effect may be inserted into the soundscape 104. Most sounds (e.g., sounds other than pure sine waves) are made up of a superposition of sinusoidal waves having different frequencies. The loudest of these frequencies is the fundamental frequency and is the sound that is the perceived by a listener as the sound's pitch. The other, quieter frequencies are referred to as overtones. Thus, inserting a fundamental frequency with a desired therapeutic effect is more effective than inserting a different sound that happens to include the frequency with the desired therapeutic as an overtone, as the therapeutic frequency will be more directly perceived by a listener.

In some embodiments, the fundamental frequency is associated with a naturally occurring frequency such as the frequency of Earth's electromagnetic field, frequencies occurring within the ocean, the frequency of a whale song, or the frequency of other natural phenomena. Association with a frequency includes the frequency itself, audible harmonics of that frequency (e.g., overtones that are integer multiples of the frequency and audible to the human ear), and/or frequencies within a just-noticeable difference (JND) of the frequency. The JND of a frequency is the smallest change from that frequency that an average human ear can detect. JND varies with baseline frequency and listening conditions (e.g., volume, tone duration, age of listener), but as a general principle, the JND is larger for higher baseline frequencies. For example, the JND for a baseline frequency of 250 Hertz (Hz) heard at a particular volume may be about 1-2 Hz, while the JND for 4000 Hz heard at the same volume may be about 15-40 Hz.

In these and other embodiments, the fundamental frequency is associated with at least one of blood pressure reduction, heart rate reduction, or a sensation of safety. For example, the fundamental frequency may be in a range of 4 Hz to 120 Hz and/or within a JND of the outer bounds (i.e., the endpoints) of that range, as these frequencies have been scientifically demonstrated to reduce blood pressure and/or heart rate of humans listening to those frequencies. As a second example, the fundamental frequency may be in a range of 7000 Hz to 12000 Hz and/or within a JND of the outer bounds of that range, as these frequencies have been scientifically demonstrated to improve a sensation of safety in patients experiencing the neurological effects (e.g., changes to an individual's mental state, such as increased empathy, decreased fear, increased emotional openness, dissociation, or hallucination) of some psychotropic substances, particularly psilocybin. As a third example, the fundamental frequency may be in a range of 15000 Hz to 30000 Hz and/or within a JND of the outer bounds of that range, as these frequencies have been scientifically demonstrated to improve a sensation of safety in patients experiencing the neurological effects of other psychotropic substances, particularly ketamine. As a fourth example, the fundamental frequency may be in a range of 4 Hz to 40 Hz, 396 Hz to 528 Hz, and/or within a JND of the outer bounds of either range, as these frequencies have been scientifically demonstrated to counteract the increased blood pressure and heart rate that commonly coincide with the neurological effects of certain psychotropic substances, particularly MDMA.

The DAW 102 may also be used to establish a particular duration for the soundscape 104 corresponding to the duration of a therapeutic session involving a particular psychotropic substance 108. The psychotropic substance 108 may be MDMA, psilocybin, ketamine, or another psychotropic substance having a neurological effect on the patient 106, who is an individual undergoing a psychotropic therapy session (e.g., undergoing an existing psychotropic therapy treatment for a mental health condition) who consumes the psychotropic substance 108. The typical durations of the neurological effects caused by different psychotropic substances differ, as do the progressions of different neurological effects caused by those substances. Thus, tailoring the duration of the soundscape 104 to the psychotropic substance 108 allows the soundscape 104 to more effectively complement the particular neurological effects of the psychotropic substance 108 as those effects progress. For example, the typical durations of the neurological effects of MDMA, psilocybin, and ketamine are about eight hours, four hours, and two hours, respectively. Consequently, when combining these durations with the fundamental frequencies for these psychotropic substances mentioned above, desirable embodiments of the soundscape distribution environment 100 may include the following:
A psychotropic substance 108 of MDMA and a soundscape 104 having a duration of eight hours and a fundamental frequency in a range of either 4 Hz to 40 Hz or 396 Hz to 528 Hz
A psychotropic substance 108 of psilocybin and a soundscape 104 having a duration of four hours and a fundamental frequency in a range of 7000 Hz to 12000 Hz
A psychotropic substance 108 of ketamine and a soundscape 104 having a duration of two hours and a fundamental frequency in a range of 15000 Hz to 30000 Hz

In some embodiments, additional features are added to the soundscape 104 via the DAW 102. For example, in addition to the fundamental frequency, a plurality of sounds may be layered within the DAW 102 (e.g., using a multi-channel layering system) such that the sounds are audible simultaneously. Layering a plurality of sounds may add a sense of weight, depth, and/or breadth to the soundscape 104 and may result in the creation of harmonic overtones with particular frequencies that are not found in any one of the sounds individually. As another example, a natural sound may be linearly stretched within the DAW 102, meaning the duration of the natural sound is increased without modifying the pitch of the natural sound. Continuing with the same example, this linear stretching may create a stretched audio track that is then inserted into the soundscape 104 via the DAW 102. This linear stretching process enables natural sounds which help induce and support therapeutic states of consciousness to be included in the soundscape 104 without retaining identifiable characteristics (e.g., the source of the sound, identifiable rhythm, identifiable transitions between frequencies) that may otherwise distract the patient 106 while listening to the soundscape 104. In these and other embodiments, the soundscape 104 is produced such that it includes no periods of total silence and/or different sounds within the soundscape 104 are stretched such that transitions between sounds are not identifiable. Thus, the soundscape 104 takes on a characteristic of constant but imperceptible change, creating a sense of progression that supports the psychotropic therapy process without distracting the patient 106.

In some embodiments, the soundscape 104 is rendered (e.g., via the DAW 102) as a binaural audio file before being provided to the patient 106. A binaural audio file is an audio file encoded to create a sensation of three-dimensional (3D) stereo sound for a listener, meaning the listener may perceive sounds from the audio file as being positioned (e.g., originating from) above, below, in front of, behind, to the left of, or to the right of a head of the listener (e.g., positioned in all three spatial dimensions). For example, the soundscape 104 may be rendered as a binaural audio file using Dolby® ATMOS® or other commercially available surround sound software. In such embodiments, the soundscape 104 may be provided to the patient 106 via headphones or via an immersive surround sound environment (e.g., a surround sound speaker system or other surround sound apparatus) to enable the patient 106 to experience the 3D stereo effect.

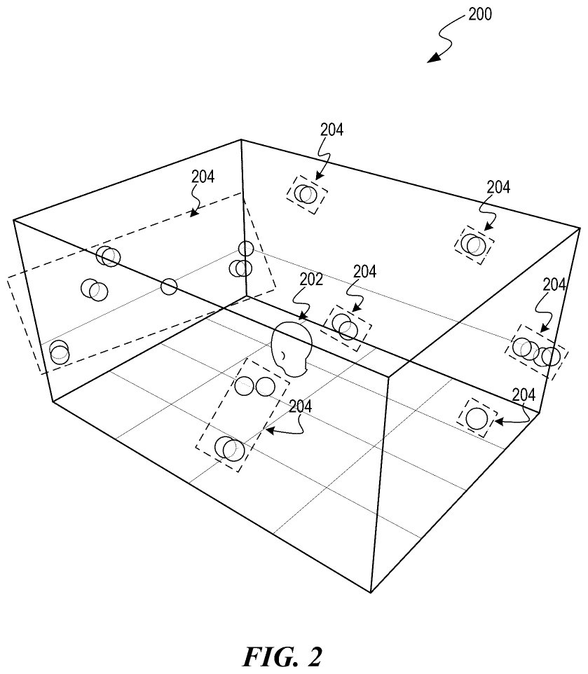

FIG. 2 is an example binaural audio plugin 200 that may be used to render (e.g., via the DAW 102) the soundscape 104 as a binaural audio file, in accordance with some embodiments of the present technology. The binaural audio plugin 200 includes a reference head 202, which represents a head of a listener to the soundscape 104. The binaural audio plugin 200 enables various sounds included in the soundscape 104 to be placed at different locations in 3D space with respect to the reference head 202. For example, as depicted in FIG. 2, the binaural audio plugin 200 includes a plurality of markers 204, which are circular visual elements that are each associated with one or more sounds in the soundscape 104. The positioning of each marker 204 relative to the reference head 202 represents the location in 3D space where the sound or sounds associated with that marker 204 will be rendered as being positioned with respect to a head of a listener to the soundscape 104. The binaural audio plugin 200 then renders those sounds such that, when the soundscape 104 is listened to, each sound is perceived as being positioned relative to the head of the listener in generally the same location as indicated by the markers 204.

### Example Digital Audio Workstation (DAW) Effects

In some embodiments, one or more audio effects are applied to the soundscape 104 using the DAW 102 before the soundscape 104 is provided to the patient 106. For example, the audio effects may include equalization (EQ), compression, saturation, peak limiting, reverb, and/or delay. Applying the one or more audio effects to the soundscape 104 may include applying the audio effect to the entire soundscape 104, to one or more particular sounds/frequencies within the soundscape 104, and/or to one or more particular temporal segments of the entire soundscape 104 and/or a particular sound/frequency therein.

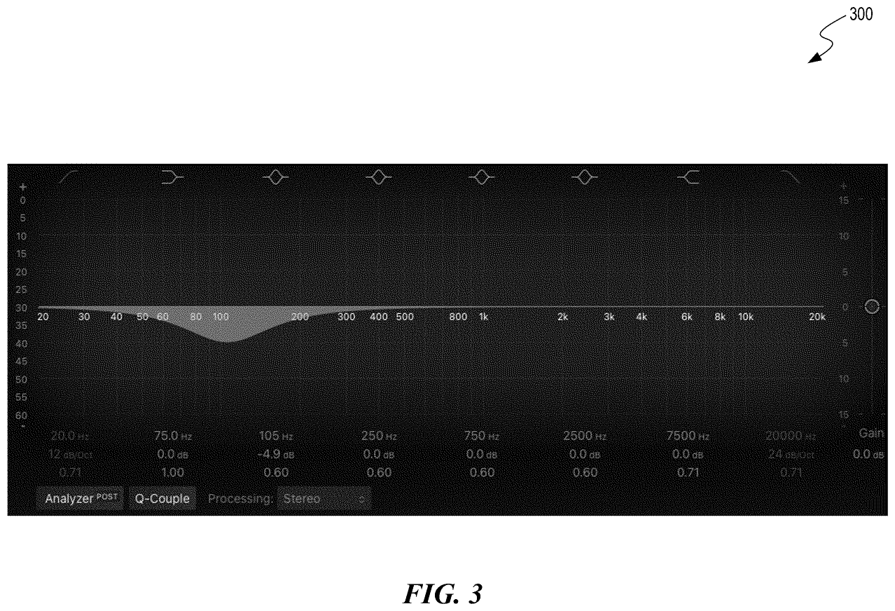

Examples of particular settings of these audio effects that may be applied to the soundscape 104 are illustrated and described in more detail in relation to FIGS. 3-10 below. The frequencies specified in relation to FIGS. 3-10 below should be understood to include the frequencies themselves as well as frequencies within a JND of the specified frequencies. Furthermore, FIGS. 3-10 are not intended to be limiting; effects may be applied to the soundscape 104 other than those illustrated in FIGS. 3-10. For example, other effects resulting in the same or a generally similar auditory output as the effects illustrated in FIGS. 3-10 may be applied to the soundscape 104, and/or a combination of one or more effects illustrated in FIGS. 3-10 may be applied.

FIG. 3 is an example EQ setting 300 that may be applied to the soundscape 104, in accordance with some embodiments of the present technology. The EQ setting 300 is configured to reduce the volume of frequencies in the soundscape of about 105 Hz most heavily, with increasingly lighter reductions in volume being applied to surrounding frequencies in a range from about 20 Hz to 700 Hz, as illustrated by the downward-facing shaded curve with a vertex at about 105 Hz.

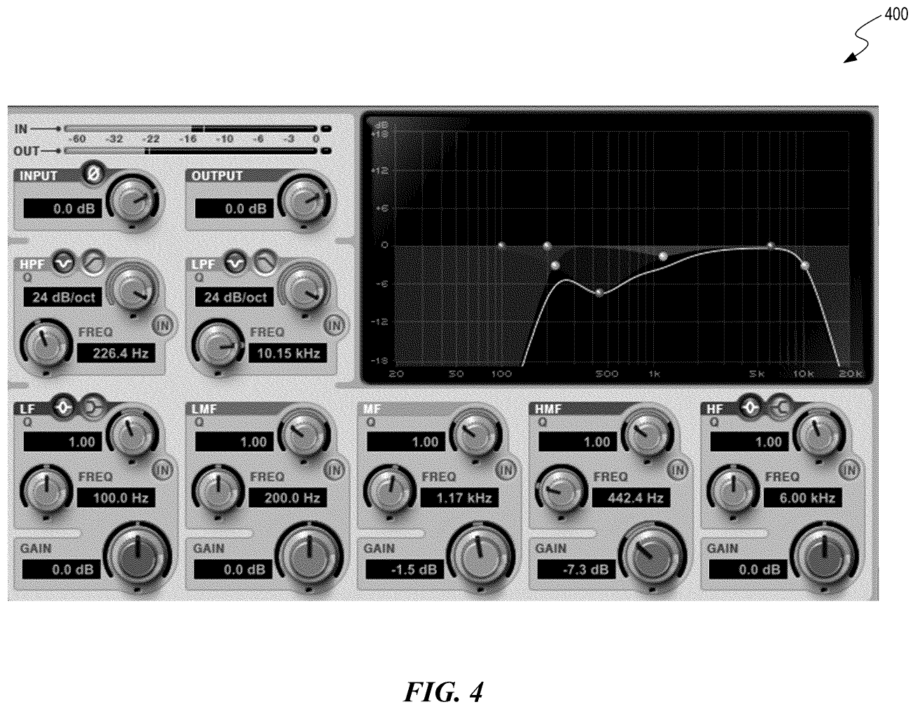

FIG. 4 is another example EQ setting, EQ setting 400, that may be applied to the soundscape 104, in accordance with some embodiments of the present technology. The EQ setting 400 is configured to apply a high-pass filter to frequencies of 226.4 Hz and below and a low-pass filter to frequencies of 10150 Hz and above. Additionally, the EQ setting 400 is configured to reduce the volume of 1170 Hz frequencies by 1.5 dB (and reduce the volume of surrounding frequencies from about 400 Hz to 4000 Hz by increasingly smaller amounts) and reduce the volume of 442.2 Hz frequencies by 7.3 dB (and reduce the volume of surrounding frequencies from about 50 Hz to 2000 Hz by increasingly smaller amounts).

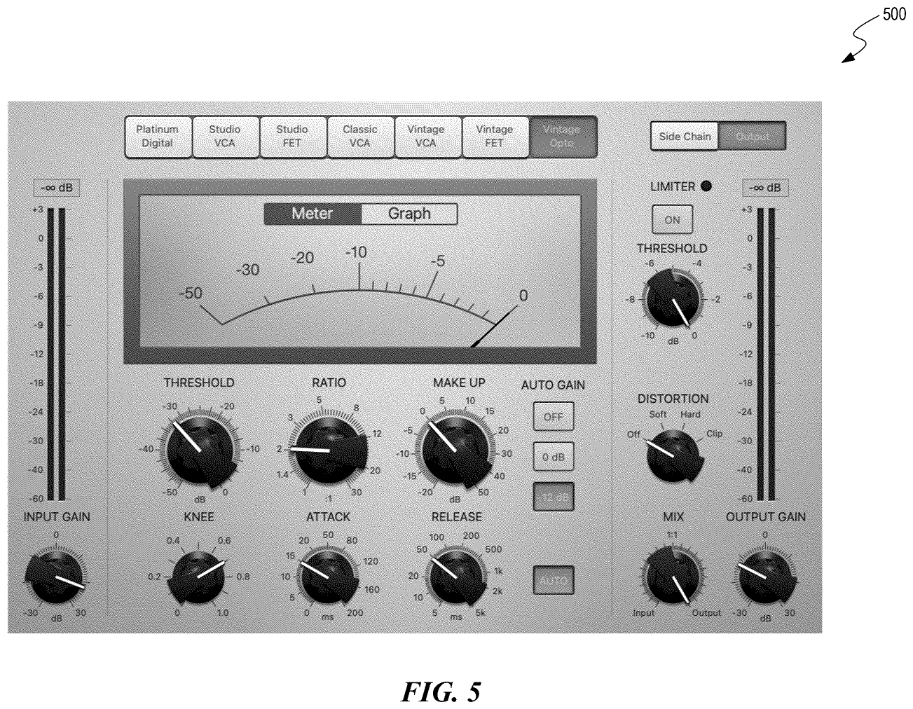

FIG. 5 is an example compressor setting 500 that may be applied to the soundscape 104, in accordance with some embodiments of the present technology. The compressor setting 500 is configured to apply an input gain of about 21 dB and an output gain of about −13 dB. Additionally, the compressor setting 500 is configured to apply a threshold of about-34 dB, a ratio of about 2.1, a makeup of about 0 dB, a knee of about 0.7, an attack of about 15 milliseconds (ms), and a release of about 50 ms.

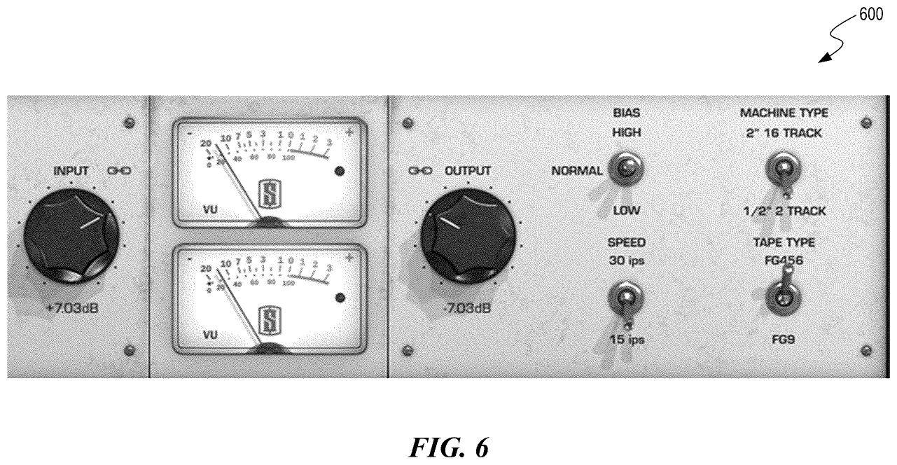

FIG. 6 is an example saturation setting 600 that may be applied to the soundscape 104, in accordance with some embodiments of the present technology. The saturation setting 600 is configured to apply an input gain of 7.03 dB, an output gain of −7.03 dB, normal bias, and a simulated tape speed of 15 inches per second (ips). Additionally, the saturation setting 600 is configured to apply tape saturation that simulates a ½ inch 2 track tape machine playing FG456 tape, thereby applying digital artifacts to the soundscape 104 that simulate the auditory quality of playing the soundscape 104 on a physical equivalent of such a tape machine/tape.

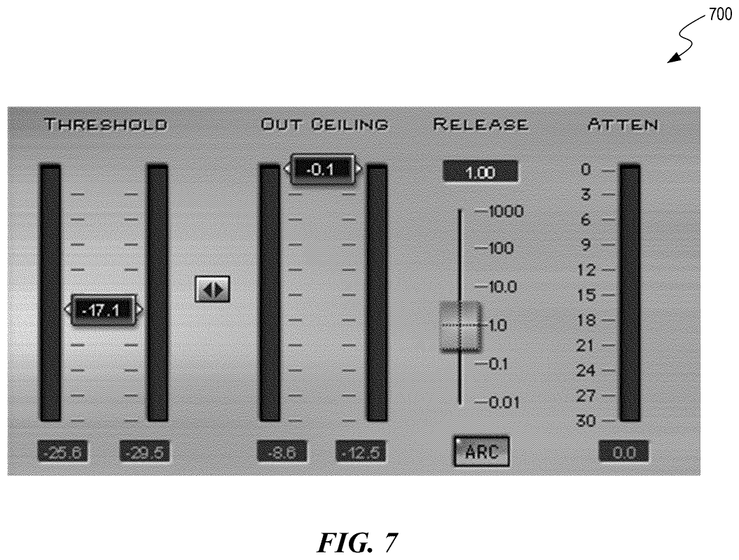

FIG. 7 is an example peak limiter setting 700 that may be applied to the soundscape 104 to limit the peak volume of sounds in the soundscape 104, in accordance with some embodiments of the present technology. The peak limiter setting 700 is configured to apply a threshold of −17.1 dB and an out ceiling (e.g., maximum peak output) of −0.1 dB. Additionally, the peak limiter setting 700 is configured to apply an automated release time, as indicated by the “ARC” (or auto-release control) symbol being highlighted.

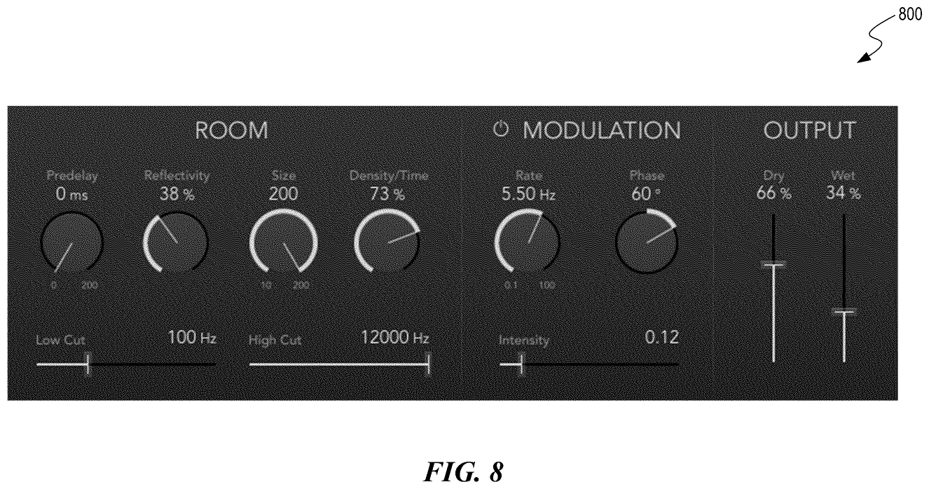

FIG. 8 is an example reverb setting 800 that may be applied to the soundscape 104, in accordance with some embodiments of the present technology. The reverb setting 800 is configured to apply room reverb with no predelay, a reflectivity of 38%, a room size of 200, a density/time of 73%, and a low-frequency oscillator (LFO) modulation rate of 5.5 Hz with a phase of 60 degrees and intensity of 0.12. Additionally, the reverb setting 800 is configured to apply a low cut of 100 Hz and a high cut of 12000 Hz, which cut frequencies below 100 Hz and above 12000 Hz, respectively, out of the generated reverb signal. The reverb setting 800 also produces an output where the dry signal (e.g., the soundscape 104 without reverb applied) is audible at 66% of maximum volume and the wet signal (e.g., the reverb signal) is audible at 34% of maximum volume.

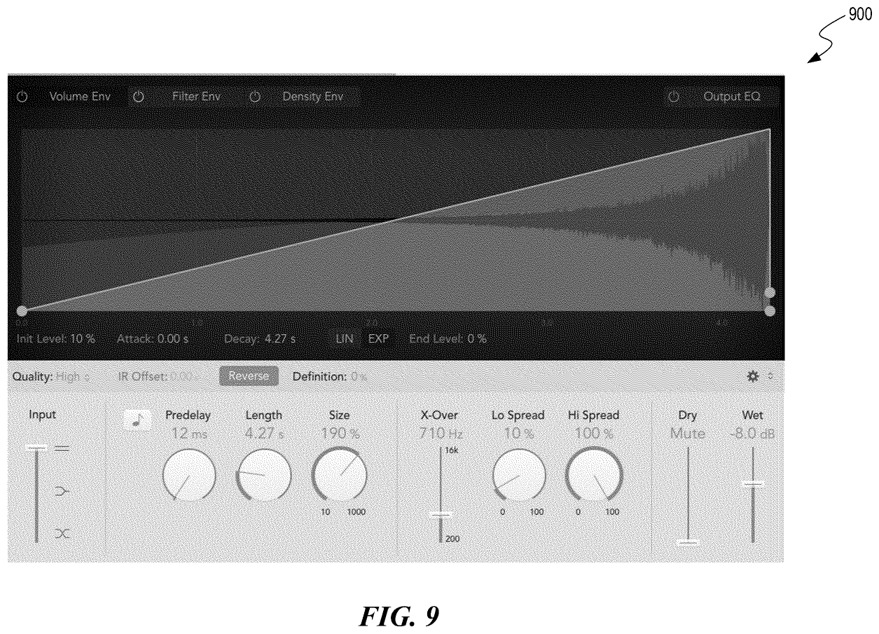

FIG. 9 is another example reverb setting, reverb setting 900, that may be applied to the soundscape 104, in accordance with some embodiments of the present technology. The reverb setting 900 is configured to apply a reversed impulse response in which reverb is applied to the tail of an input signal (e.g., the soundscape 104 or an individual sound within the soundscape 104) rather than to the front of the input signal. Additionally, the reverb setting 900 is configured to apply a predelay of 12 ms, a length of 4.27 seconds, a size of 190%, a crossover frequency (X-over) of 710 Hz, a spread of 10% (lo spread) to frequencies below the crossover frequency, and a spread of 100% (hi spread) to frequencies above the crossover frequency. The reverb setting 900 also produces an output where the dry signal is entirely muted and the output volume of the wet signal is reduced by 8 dB.

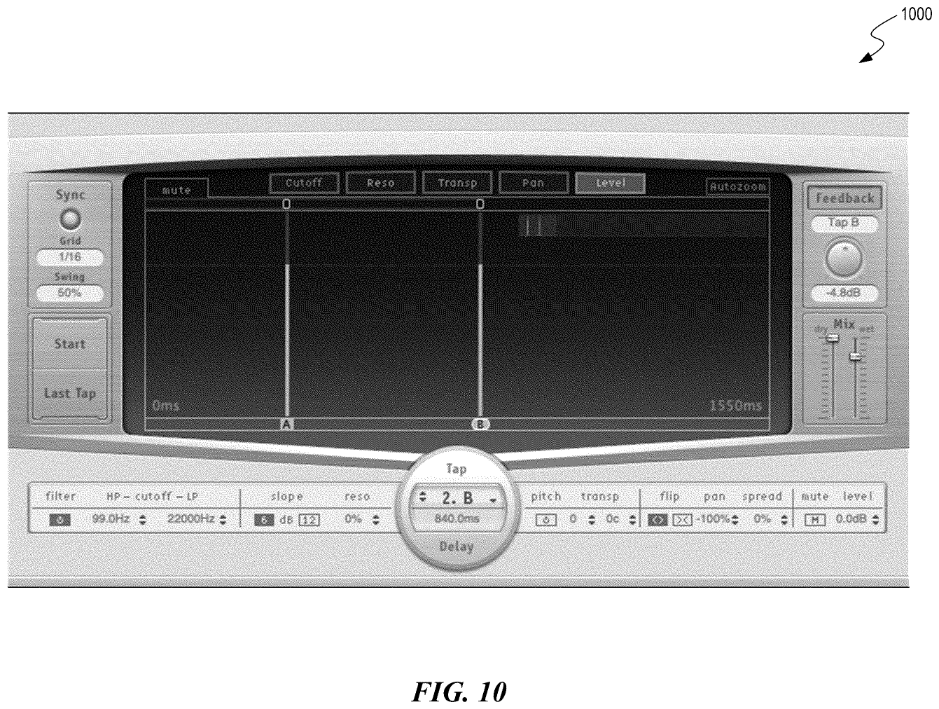

FIG. 10 is an example delay setting 1000 that may be applied to the soundscape 104, in accordance with some embodiments of the present technology. The delay setting 1000 is configured to apply two delay taps to an input signal (e.g., the soundscape 104 or an individual sound within the soundscape 104), one at about 375 ms after receiving the input signal (labeled tap “A”), and another at 840 ms after receiving the input signal (labeled tap “B”). The delay taps are configured to be of equal volume, as indicated by the shaded bars extending the same vertical distance from both tap A and tap B. Additionally, the delay setting 1000 is configured to apply a high-pass filter to frequencies of 99 Hz and below and a low-pass filter to frequencies of 22000 Hz and above.

### Example Method Flowcharts

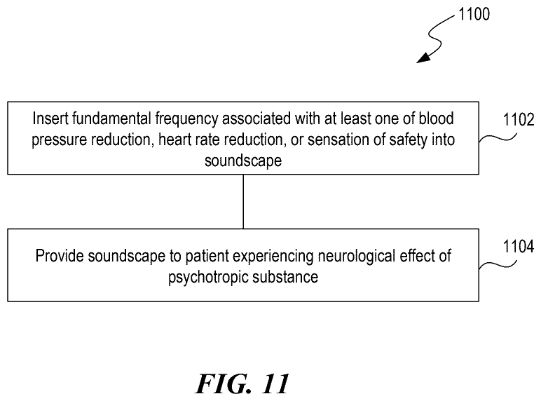

FIG. 11 is a flowchart depicting an example method 1100 of providing a soundscape to a patient, in accordance with some embodiments of the present technology. In some embodiments, the method 1100 is performed by components of the example soundscape distribution environment 100 described in relation to FIG. 1 above. Likewise, embodiments can include different and/or additional operations or can perform the operations in different orders.

At operation 1102, a fundamental frequency associated with at least one of blood pressure reduction, heart rate reduction, or sensation of safety is inserted into a soundscape. For example, the soundscape may be the same as or generally similar to the soundscape 104 described in relation to FIG. 1 above and therefore lack traditional musical elements such as identifiable musical instrumentation, identifiable vocalizations, identifiable harmonic structure, identifiable rhythm, and/or identifiable tempo. In some embodiments, the fundamental frequency is within one of the frequency ranges described in relation to FIG. 1 above, or within a JND of the outer bounds of one of those frequency ranges. In these and other embodiments, the fundamental frequency is inserted using a DAW (e.g., a DAW 102 as described in relation to FIG. 1 above).

At operation 1104, the soundscape is provided to a patient experiencing a neurological effect of a psychotropic substance. For example, the psychotropic substance may be the same as or generally similar to the psychotropic substance 108 described in relation to FIG. 1 above, and thus may be a psychotropic substance such as MDMA, psilocybin, or ketamine. In some embodiments, the soundscape is rendered as a binaural audio file and provided via headphones worn by the patient or via an immersive surround sound environment, enabling the patient to experience the 3D stereo effect of the binaural audio. The patient may be a patient 106 as described in relation to FIG. 1 above.

In some embodiments, before the soundscape is provided to the patient, additional modifications are made to the soundscape (e.g., via a DAW). For example, a natural sound may be linearly stretched to create a stretched audio track and the stretched audio track may be inserted into the soundscape. As another example, at least one of equalization, compression, saturation, peak limiting, reverb, or delay may be applied to the soundscape. As a third example, a plurality of sounds may be layered within the soundscape such that the sounds are audible simultaneously.

In some embodiments, the psychotropic substance is MDMA, the soundscape has a duration of eight hours, and the fundamental frequency is either in a range of 4 Hz to 40 Hz, in a range of 396 Hz to 528 Hz, or within a JND of an outer bound of either range. In other embodiments, the psychotropic substance is psilocybin, the soundscape has a duration of four hours, and the fundamental frequency is either in a range of 7000 Hz to 12000 Hz or within a JND of an outer bound of that range. In still other embodiments, the psychotropic substance is ketamine, the soundscape has a duration of two hours, and the fundamental frequency is either in a range of 15000 Hz to 30000 Hz or within a JND of an outer bound of that range.

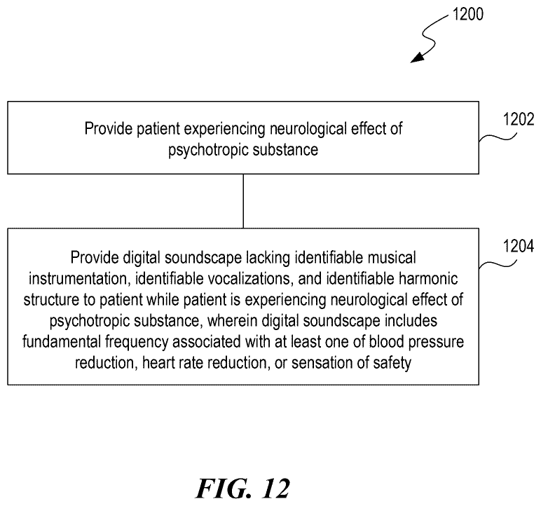

FIG. 12 is a flowchart depicting an example psychotropic therapy method 1200, in accordance with some embodiments of the present technology. In some embodiments, the psychotropic therapy method 1200 is performed by components of the example soundscape distribution environment 100 described in relation to FIG. 1 above. Likewise, embodiments can include different and/or additional operations or can perform the operations in different orders.

At operation 1202, a patient experiencing a neurological effect of a psychotropic substance is provided. For example, the psychotropic substance may be the same as or generally similar to the psychotropic substance 108 described in relation to FIG. 1 above, and thus may be a psychotropic substance such as MDMA, psilocybin, or ketamine. The patient may be a patient 106 as described in relation to FIG. 1 above.

At operation 1204, a digital soundscape lacking identifiable musical instrumentation, identifiable vocalizations, and identifiable harmonic structure is provided to the patient while the patient is experiencing the neurological effect of the psychotropic substance. The digital soundscape includes a fundamental frequency associated with at least one of blood pressure reduction, heart rate reduction, or a sensation of safety. For example, the fundamental frequency may be within one of the frequency ranges described in relation to FIG. 1 above, or within a JND of the outer bounds of one of those frequency ranges. In some embodiments, the soundscape is the same as or generally similar to the soundscape 104 described in relation to FIG. 1 above.

In some embodiments, the psychotropic substance is MDMA, the soundscape has a duration of eight hours, and the fundamental frequency is either in a range of 4 Hz to 40 Hz, in a range of 396 Hz to 528 Hz, or within a JND of an outer bound (i.e., one of the ends) of either range. In other embodiments, the psychotropic substance is psilocybin, the soundscape has a duration of four hours, and the fundamental frequency is either in a range of 7000 Hz to 12000 Hz or within a JND of an outer bound of that range. In still other embodiments, the psychotropic substance is ketamine, the soundscape has a duration of two hours, and the fundamental frequency is either in a range of 15000 Hz to 30000 Hz or within a JND of an outer bound of that range.

### CONCLUSION

Alternative language and synonyms can be used for any one or more of the terms discussed herein, and no special significance is to be placed upon whether or not a term is elaborated or discussed herein. Synonyms for certain terms are provided. A recital of one or more synonyms does not exclude the use of other synonyms. The use of examples anywhere in this specification, including examples of any term discussed herein, is illustrative only and is not intended to further limit the scope and meaning of the disclosure or of any exemplified term. Likewise, the disclosure is not limited to various embodiments given in this specification.

It is to be understood that the embodiments and variations shown and described herein are merely illustrative of the principles of this invention and that various modifications can be implemented by those skilled in the art.

Note that any and all of the embodiments described above can be combined with each other, except to the extent that it may be stated otherwise above or to the extent that any such embodiments might be mutually exclusive in function and/or structure.

Although the present invention has been described with reference to specific exemplary embodiments, it will be recognized that the invention is not limited to the embodiments described but can be practiced with modification and alteration within the spirit and scope of the appended claims. Accordingly, the specification and drawings are to be regarded in an illustrative sense rather than a restrictive sense.

I/We claim:

1. A method of distributing a digitally produced soundscape lacking identifiable musical instrumentation, identifiable vocalizations, and identifiable harmonic structure, the method comprising:
inserting a fundamental frequency associated with at least one of blood pressure reduction, heart rate reduction, or a sensation of safety into the soundscape; and
providing the soundscape to a patient experiencing a neurological effect of a psychotropic substance.

2. The method of claim 1, wherein the psychotropic substance is 3,4-Methylenedioxymethamphetamine (MDMA), wherein the soundscape has a duration of eight hours, and wherein the fundamental frequency is either (i) in a range of 4 Hz to 40 Hz, (ii) in a range of 396 Hz to 528 Hz, or (iii) within a just-noticeable difference (JND) of an outer bound of either range.

3. The method of claim 1, wherein the psychotropic substance is psilocybin, wherein the soundscape has a duration of four hours, and wherein the fundamental frequency is either (i) in a range of 7000 Hz to 12000 Hz or (ii) within a JND of an outer bound of the range.

4. The method of claim 1, wherein the psychotropic substance is ketamine, wherein the soundscape has a duration of two hours, and wherein the fundamental frequency is either (i) in a range of 15000 Hz to 30000 Hz or (ii) within a JND of an outer bound of the range.

5. The method of claim 1, wherein the soundscape is provided either via headphones worn by the patient or via an immersive surround sound environment, the method further comprising:
rendering the soundscape as a binaural audio file.

6. The method of claim 1, further comprising:
linearly stretching a natural sound to create a stretched audio track; and
inserting the stretched audio track into the soundscape.

7. The method of claim 1, further comprising:
applying at least one of equalization, compression, saturation, peak limiting, reverb, or delay to the soundscape.

8. The method of claim 1, wherein the soundscape includes a plurality of sounds layered to be audible simultaneously.

9. The method of claim 1, wherein the soundscape further lacks an identifiable tempo and an identifiable rhythm.

10. A psychotropic therapy method comprising:
providing a patient experiencing a neurological effect of a psychotropic substance; and
providing a digital soundscape lacking identifiable musical instrumentation, identifiable vocalizations, and identifiable harmonic structure to the patient while the patient is experiencing the neurological effect of the psychotropic substance, wherein the digital soundscape includes a fundamental frequency associated with at least one of blood pressure reduction, heart rate reduction, or a sensation of safety.

11. The method of claim 10, wherein the psychotropic substance is 3,4-Methylenedioxymethamphetamine (MDMA), wherein the soundscape has a duration of eight hours, and wherein the fundamental frequency is either (i) in a range of 4 Hz to 40 Hz, (ii) in a range of 396 Hz to 528 Hz, or (iii) within a just-noticeable difference (JND) of an outer bound of either range.

12. The method of claim 10, wherein the psychotropic substance is psilocybin, wherein the soundscape has a duration of four hours, and wherein the fundamental frequency is either (i) in a range of 7000 Hz to 12000 Hz or (ii) within a JND of an outer bound of the range.

13. The method of claim 10, wherein the psychotropic substance is ketamine, wherein the soundscape has a duration of two hours, and wherein the fundamental frequency is either (i) in a range of 15000 Hz to 30000 Hz or (ii) within a JND of an outer bound of the range.

14. The method of claim 10, wherein the soundscape is a binaural audio file provided to the patient either via headphones worn by the patient or via an immersive surround sound environment.

15. The method of claim 10, wherein the soundscape further lacks an identifiable tempo and an identifiable rhythm.

16. A digitally produced soundscape for augmenting therapeutic neurological effects of a psychotropic substance, wherein the soundscape lacks identifiable musical instrumentation, identifiable vocalizations, and identifiable harmonic structure, the soundscape comprising:
a fundamental frequency associated with at least one of blood pressure reduction, heart rate reduction, or a sensation of safety.

17. The soundscape of claim 16, wherein the psychotropic substance is 3,4-Methylenedioxymethamphetamine (MDMA), wherein the soundscape has a duration of eight hours, and wherein the fundamental frequency is either (i) in a range of 4 Hz to 40 Hz, (ii) in a range of 396 Hz to 528 Hz, or (iii) within a just-noticeable difference (JND) of an outer bound of either range.

18. The soundscape of claim 16, wherein the psychotropic substance is psilocybin, wherein the soundscape has a duration of four hours, and wherein the fundamental frequency is either (i) in a range of 7000 Hz to 12000 Hz or (ii) within a JND of an outer bound of the range.

19. The soundscape of claim 16, wherein the psychotropic substance is ketamine, wherein the soundscape has a duration of two hours, and wherein the fundamental frequency is either (i) in a range of 15000 Hz to 30000 Hz or (ii) within a just-noticeable difference (JND) of an outer bound of the range.

20. The soundscape of claim 16, further comprising a linearly stretched natural sound.
